(19) United States
(12) Patent Application Publication
(10) Pub. No.: US 2005/0165423 A1
(43) Pub. Date: Jul. 28, 2005

(54) Ligating clip with integral tissue-securing mechanism

(75) Inventors: Richard J. Gallagher, Raleigh, NC (US); Lowell M. LaFreniere, Raleigh, NC (US); Tu Tran, Apex, NC (US)

Correspondence Address: JENKINS, WILSON & TAYLOR, P. A., 3100 TOWER BLVD, SUITE 1400, DURHAM, NC, 27707, US

(73) Assignee: Pilling Weck Incorporated

(21) Appl. No.: 10/763,439

(22) Filed: Jan. 23, 2004

(51) Int. Cl. A61B 17/08

(52) U.S. Cl. 606/151

(57) ABSTRACT

A polymeric, surgical clip having first and second curved leg members joined at their proximal end by a hinge portion and movable from an open position to a closed position for clamping a vessel between curved opposing inner surfaces which are substantially parallel when the clip is closed. An interlocking mechanism is formed by a portion of the inner surfaces of the first and second legs. The interlocking mechanism may be a tongue-in-groove mechanism, formed by a lip or tongue protruding from a portion of the inner surface of one leg and a groove formed in a corresponding portion of the inner surface of the other leg, or a lock-step mechanism, formed by complementary L-shaped notches wherein a notch is provided in a portion of the inner surface of each leg. The interlocking mechanism acts to impede longitudinal movement of the clip relative to the vessel being clamped.

### TECHNICAL FIELD

The present invention relates to surgical clips, and more particularly to ligating clips with an integral tissue-securing mechanism to impede the longitudinal movement of the ligating clip along a vessel engaged by the clip. Yet more particularly, the present invention relates to an improved surgical ligating clip that is provided with an interlocking mechanism integral to the legs of the clip that serve to secure the tissue or vessel engaged by the clip.

### BACKGROUND ART

Many surgical procedures require vessels or other tissues of the human body to be ligated during the surgical process. For example, many surgical procedures require cutting blood vessels (e.g., veins or arteries), and these blood vessels may require ligation to reduce bleeding. In some instances, a surgeon may wish to ligate the vessel temporarily to reduce blood flow to the surgical site during the surgical procedure. In other instances a surgeon may wish to permanently ligate a vessel. Ligation of vessels or other tissues can be performed by closing the vessel with a ligating clip, or by suturing the vessel with surgical thread. The use of surgical thread for ligation requires complex manipulations of the needle and suture material to form the knots required to secure the vessel. Such complex manipulations are time-consuming and difficult to perform, particularly in endoscopic surgical procedures, which are characterized by limited space and visibility. By contrast, ligating clips are relatively easy and quick to apply. Accordingly, the use of ligating clips in endoscopic as well as open surgical procedures has grown dramatically.

Various types of hemostatic and aneurysm clips are used in surgery for ligating blood vessels or other tissues to stop the flow of blood. Such clips have also been used for interrupting or occluding ducts and vessels in particular surgeries such as sterilization procedures. Typically, a clip is applied to the vessel or other tissue by using a dedicated mechanical instrument commonly referred to as a surgical clip applier, ligating clip applier, or hemostatic clip applier. Generally, the clip is left in place after application to the tissue until hemostasis or occlusion occurs. At some point thereafter, the clip is removed by using a separate instrument dedicated for that purpose, i.e., a clip removal instrument.

Ligating clips can be classified according to their geometric configuration (e.g., symmetric clips or asymmetric clips), and according to the material from which they are manufactured (e.g., metal clips or polymeric clips). Symmetric clips are generally “U” or “V” shaped and thus are substantially symmetrical about a central, longitudinal axis extending between the legs of the clip. Symmetric clips are usually constructed from metals such as stainless steel, titanium, tantalum, or alloys thereof. By means of a dedicated clip applier, the metal clip is permanently deformed over the vessel. An example of one such clip is disclosed in U.S. Pat. No. 5,509,920 to Phillips et al. An example of a metallic clip applier is disclosed in U.S. Pat. No. 3,326,216 to Wood in which a forceps-type applier having conformal jaws is used to grip and maintain alignment of the clip during deformation. Such appliers may additionally dispense a plurality of clips for sequential application, as disclosed in U.S. Pat. No. 4,509,518 to McGarry et al.

With the advent of high technology diagnostic techniques using computer tomography (CATSCAN) and magnetic resonance imaging (MRI), metallic clips have been found to interfere with the imaging techniques. To overcome such interference limitations, biocompatible polymers have been increasingly used for surgical clips. Unlike metallic clips, which are usually symmetric, polymeric clips are usually asymmetric in design and hence lack an axis of symmetry. Inasmuch as the plastic clip cannot be permanently deformed for secure closure around a vessel or other tissue, latching mechanisms have been incorporated into the clip design to establish closure conditions and to secure against re-opening of the vessel. For example, well known polymeric clips are disclosed in U.S. Pat. No. 4,834,096 to Oh et al. and U.S. Pat. No. 5,062,846 to Oh et al., both of which are assigned to the assignee of the present invention. These plastic clips generally comprise a pair of curved legs joined at their proximal ends with an integral hinge or heel. The distal ends of the curved legs include interlocking latching members. For example, the distal end of one leg terminates in a lip or hook structure into which the distal end of the other leg securely fits to lock the clip in place.

The distal ends of the clips taught by Oh et al. also include lateral bosses that are engaged by the jaws of the clip applier. A clip applier specifically designed for asymmetric plastic clips is used to close the clip around the tissue to be ligated, and to latch or lock the clip in the closed condition. In operation, the jaws of this clip applier are actuated into compressing contact with the legs of the clip. This causes the legs to pivot inwardly about the hinge, thereby deflecting the hook of the one leg to allow reception therein of the distal end of the other leg. A clip applier designed for use with asymmetric plastic clips in an open (i.e., non-endoscopic) surgical procedure is disclosed in U.S. Pat. No. 5,100,416 to Oh et al., also assigned to the assignee of the present invention.

In addition to compatibility with sophisticated diagnostic techniques, asymmetric clips have other advantages over symmetric clips. For example, because asymmetric clips are formed from polymeric materials, the mouths of asymmetric clips can be opened wider than the mouths of symmetric clips. This allows a surgeon to position the clip about the desired vessel with greater accuracy. In addition, a clip of the type described in U.S. Pat. Nos. 4,834,096 and 5,062,846 to Oh et al. can be repositioned before locking the clip on the vessel or before removing the clip from the vessel, in a process referred to as “approximating” the clip.

Although plastic ligating clips are well known in the surgical area and improvements have been made to the ligating clips, including providing protrusions on the inner surfaces of the leg members to impede the lateral movement of a vessel during clip closure (see, for example, the aforementioned U.S. Pat. Nos. 4,834,096 and 5,062,846), these improvements have been less effective in preventing longitudinal movement of a vessel or tissue during and after clip closure. Accordingly, there is a need to provide an improved polymeric surgical ligating clip with an interlocking mechanism integral to the legs of the clip that serves to secure the tissue or vessel engaged by the clip.

### SUMMARY OF THE INVENTION

In accordance with the present invention, a polymeric surgical clip is provided of the type comprising first and second leg members joined at their proximal ends by a resilient hinge means. Each leg member has a vessel clamping inner surface and an opposite outer surface, and the vessel clamping inner surface is in opposition to the vessel clamping inner surface of the other leg member. The first leg member terminates at its distal end in a deflectable hook member curved toward the second leg member, and the second leg member terminates at its distal end in a locking portion complimentary to the hook member such that when the first and second leg members are moved from an open position to a closed position about the hinge means, the hook member deflects about the distal end of the second leg member to lock the clip in a closed position. The hook member has a continuously curved outer surface extending distally from the outer surface of the first leg member, side surfaces and an inner surface.

The improvement to the polymeric surgical clip comprises providing an interlock mechanism comprising complimentary parts formed along a portion of the vessel clamping inner surface of each of the first and second leg members. The complementary parts cooperatively engage when the clip is in the closed position to capture a vessel or other tissue and impede longitudinal movement of the clip in relation to the vessel or other tissue.

The surgical clip of the present invention is preferably made of polymeric material and accordingly minimizes interference with high technology diagnostic modalities such as CAT SCAN, MRI and MRS. At the same time, the clip is nearly as small as comparable metal clips while maintaining sufficient strength and possessing high security in the clip's latching mechanism in the closed position clamping the vessel. The surgical clip is configured to provide a secure means of handling an application to avoid premature release from the applier of the clip.

It is therefore an object of the present invention to provide a polymeric surgical clip capable of occluding a vessel while resisting longitudinal movement along the vessel.

It is another object of the present invention to provide a surgical clip with an interlocking tissue-securing mechanism integral to the inner surfaces of the legs of the clip that serves to secure the clip to the vessel and prevent longitudinal movement of the clip relative to the vessel when the clip is in the closed position.

Some of the objects of the invention having been stated hereinabove, other objects will become evident as the description proceeds when taken in connection with the accompanying drawings as best described hereinbelow.

### BRIEF DESCRIPTION OF THE DRAWINGS

FIG. 1 is an enlarged perspective view of the surgical ligating clip of the present invention;

FIG. 2 is an enlarged side elevation view of the surgical ligating clip of the present invention;

FIG. 3 is an enlarged, partially sectioned view of the surgical ligating clip viewed along line 3-3 in FIG. 2;

FIG. 4 shows the clip of FIG. 1 applied to a body vessel;

FIG. 5 is a cross-sectional view of the clip applied to a body vessel viewed along line 5-5 in FIG. 4;

FIG. 6 is an enlarged perspective view of an alternate embodiment of the surgical ligating clip of the present invention;

FIG. 7 is an enlarged side elevation view of the alternate embodiment of the surgical ligating clip of the present invention;

FIG. 8 is an enlarged, partially sectioned view of the alternate embodiment of the surgical ligating clip viewed along line 8-8 in FIG. 7;

FIG. 9 shows the clip of FIG. 6 applied to a body vessel; and

FIG. 10 is a cross-sectional view of the clip applied to a body vessel viewed along line 10-10 in FIG. 9.

### DETAILED DESCRIPTION OF THE INVENTION

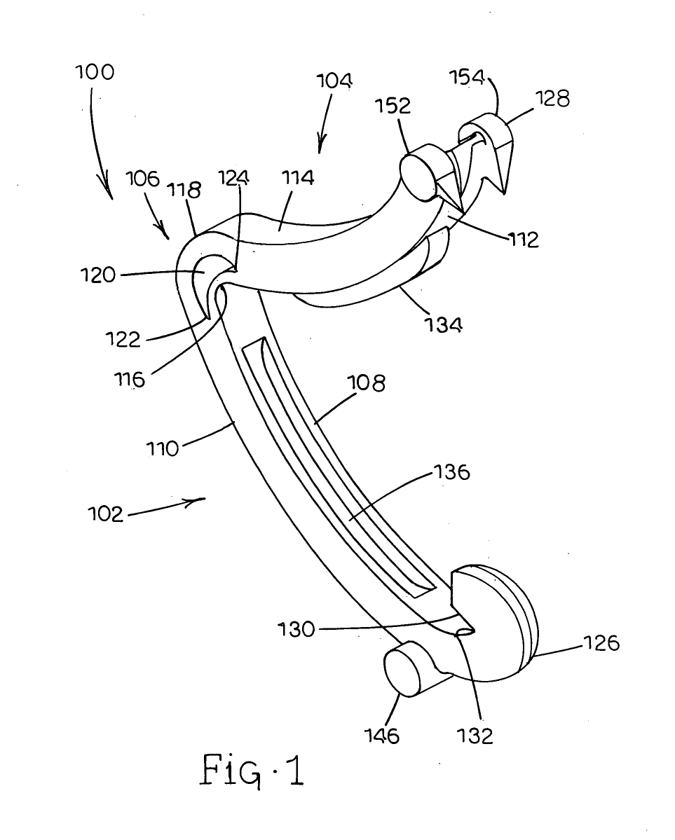

Referring to FIG. 1 through FIG. 3, an example is illustrated of an asymmetric surgical clip generally designated 100 that is suitable for use in conjunction with the present invention. Clip 100 and others of similar design are particularly useful as hemostatic clips that can be latched around a vessel or other type of tissue to ligate the vessel and thereby stop or reduce the flow of fluid through the vessel. Clip 100 can be constructed from any suitable biocompatible material, such as certain metals and polymers. However, the present invention is particularly suitable for practice with polymeric clips. Thus, clip 100 preferably comprises a one-piece integral polymeric body formed from a suitable strong biocompatible engineering plastic such as the type commonly used for surgical implants. Examples include polyethylene terephthalate (PET), polybutylene terephthalate (PBT), polyoxymethylene, or other thermoplastic materials having similar properties that can be injection-molded, extruded or otherwise processed into like articles.

FIG. 1 is an enlarged perspective view of the surgical ligating clip of the present invention. The body of clip 100 includes a first or outer leg, generally designated 102, and a second or inner leg, generally designated 104. First and second legs 102 and 104 are joined at their proximal ends by an integral hinge section, generally designated 106. First and second legs 102 and 104 have complementary arcuate profiles. Thus, first leg 102 has a concave inner surface 108 and a convex outer surface 110, and second leg 104 has a convex inner surface 112 and a concave outer surface 114. Convex inner surface 112 of second leg 104 and concave inner surface 108 of first leg 102 have substantially matching radii of curvature.

Hinge section 106 has a continuous concave inner surface 116 and a continuous convex outer surface 118. Concave inner surface 116 of hinge section 106 joins concave inner surface 108 of first leg 102 and convex inner surface 112 of second leg 104. Convex outer surface 118 of hinge section 106 joins convex outer surface 110 of first leg 102 and concave outer surface 114 of second leg 104. Curved slot 120 is located between curved hinge surfaces 116 and 118, and is positioned closer to inner surface 116 than to outer surface 118. Slot 120 extends completely through hinge section 106 from side to side and its opposite ends 122, 124 extend into the proximal ends of first and second legs 102 and 104, respectively. Slot 120 provides added flexibility to hinge section 106, but the inner concave surface 116 prevents any portion of the clamped vessel from being trapped within slot 120.

First leg 102 transitions to a curved, C-shaped hook section 126 at its distal end. Second leg 104 transitions to a pointed tip section 128 at its distal end. The distal portion of hook section 126 curves inwardly and points generally toward inner surface 116 of hinge 106. The hook section has a transverse beveled surface 130 and a concave inner surface 108 that defines a latching recess 132. The latching recess 132 is adapted for conformally engaging tip section 128 in the course of compressing clip 100 into a latched or locked position around a vessel or other tissue.

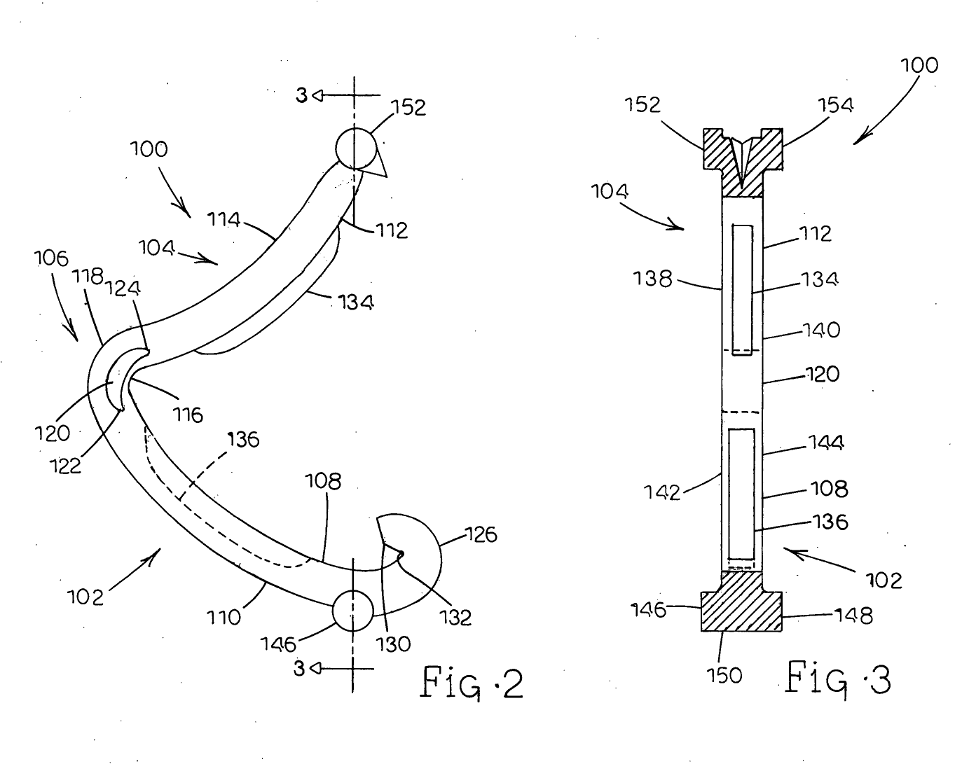

In accordance with the invention, raised lip or tongue 134 protrudes from inner surface 112 of second leg 104. Tongue 134 is oriented longitudinally along a portion of inner surface 112 of second leg 104. As shown in FIG. 2, which is an enlarged side elevation view of the surgical ligating clip of the present invention, the proximal and distal ends of tongue 134 may be curved, giving tongue 134 a generally oval or elliptical profile. Recessed pocket or groove 136 is formed longitudinally along a portion of inner surface 108 of first leg 102. Groove 136 has a profile complementary to tongue 134 and is positioned opposite to tongue 134. Tongue 134 and groove 136 form complementary parts of an interlocking mechanism. Accordingly, when clip 100 is compressed into a latched or locked position, tongue 134 fits within groove 136. The curved ends of tongue 134 reduce the likelihood that tissue captured in clip 100 will be damaged by tongue 134. One would appreciate that groove 136 should be larger than tongue 134 to accommodate tongue 134 and the portion of any vessel or tissue captured by clip 100 along tongue 134.

As best shown in FIG. 3, which is a view directed into the open concave side of clip 100 viewed along line 3-3 in FIG. 2, clip 100 has parallel, opposed side surfaces. Tongue 134 is approximately centered between side surfaces 138 and 140 of second leg member 104. Similarly, groove 136 is approximately centered between side surfaces 142 and 144 of first leg member 102. By centering groove 136 between side surfaces 142 and 144, approximately equal amounts of clip material are on each of the lateral sides of groove 136 and help secure the captured tissue. The width and length of tongue 134 are smaller than the width and length of groove 136. As noted above, the larger dimensions of groove 136 permit an amount of tissue to be pushed into groove 136 by tongue 134. The larger dimensions of groove 136 also aid in the alignment of tongue 134 and groove 136 while clip 100 is being compressed by permitting a certain amount of play in the alignment of first leg 102 and second leg 104.

Adjacent to the distal end of the first leg 102 and immediately inward of hook section 126, cylindrical bosses 146 and 148 protrude perpendicular to each of the opposed side surfaces 142 and 144. In the illustrated example of clip 100, a bridge section 150 couples bosses 146 and 148 together. As evident in FIG. 2, bosses 146 and 148 project outwardly beyond convex outer surface 110 of first leg 102. At the distal end of second or inner leg 104, cylindrical bosses 152 and 154 protrude perpendicular to each of the opposed side surfaces 138 and 140 of inner leg 104 at tip section 128. Bosses 152 and 154 of second leg 104 extend longitudinally forwardly beyond tip section 128.

In the practice of ligating a vessel as understood by persons skilled in the art, clip 100 is designed to be compressed into a latched or locked position around the vessel through the use of an appropriate clip applicator instrument, such as the type described in the aforementioned U.S. Pat. No. 5,100,416. The clip applicator instrument engages bosses 146, 148, 152 and 154 of clip 100 and pivots bosses 146, 148, 152 and 154 inwardly about hinge section 106. This causes first and second legs 102 and 104 to close around the vessel, with convex inner surface 112 of second leg 104 and complementary concave inner surface 108 of first leg 102 contacting the outer wall of the vessel. Tongue 134 pushes a portion of the vessel into groove 136. Tongue 134 and groove 136 effectively secure the clip to the vessel and prevent longitudinal movement of the clip or vessel during or after clip closure. Tip section 128 of second leg 104 then begins to contact hook section 126. Further pivotal movement by the applicator instrument longitudinally elongates first leg 102 and deflects hook section 126, allowing tip section 128 to align with latching recess 132. Upon release of the applicator instrument, tip section 128 snaps into and is conformably seated in latching recess 132, at which point clip 100 is in its latched condition and the vessel securely engaged thereby.

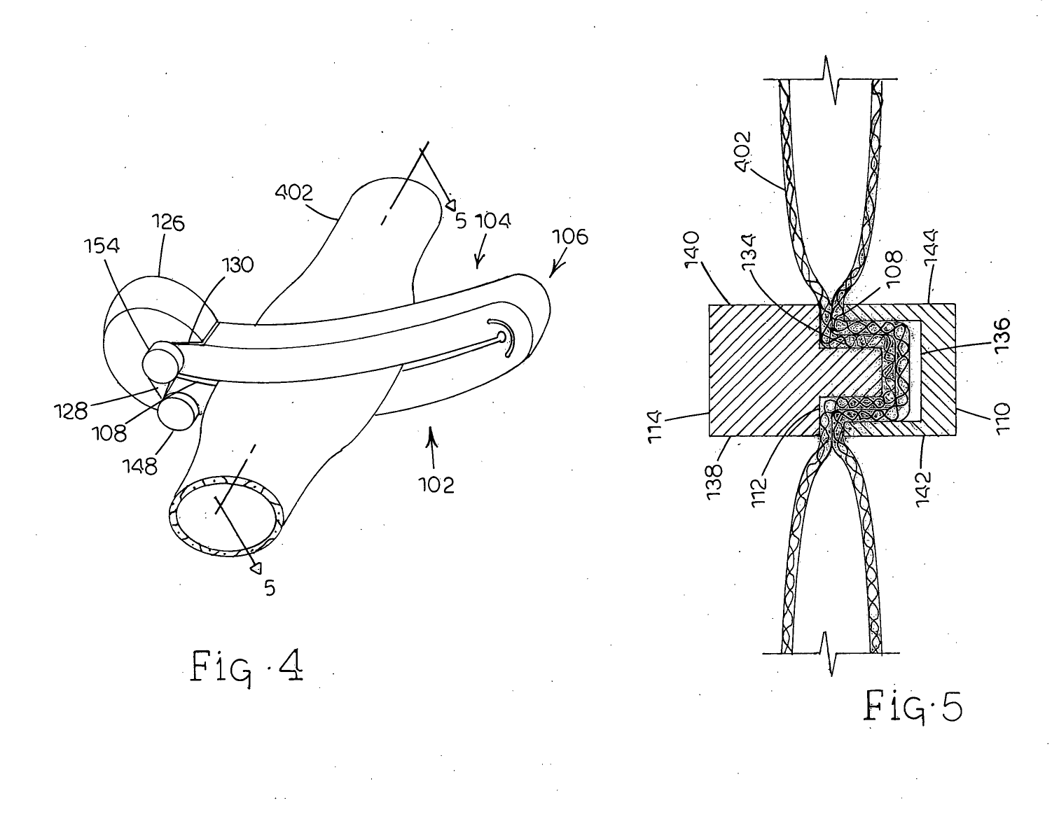

FIG. 4 is an enlarged, perspective view of clip 100 compressed around a portion of a vessel. In the latched condition, tip section 128 is engaged between concave inner surface 108 and beveled surface 130, thereby securely clamping a designated vessel or other tissue between concave inner surface 108 and convex inner surface 112.

FIG. 5 is a cross sectional view of clip 100 engaged around a portion of a vessel as viewed along line 5-5 in FIG. 4. In the area where clip 100 is applied to the vessel, tongue 134 makes contact with a portion of the vessel and pushes the vessel into groove 136. The portion of the vessel in contact with tongue 134 conforms around tongue 134 as the vessel is pushed into groove 136. Longitudinal movement of the vessel relative to clip 100 is resisted primarily by the portion of the vessel trapped between the sides of tongue 134 and the sides of groove 136.

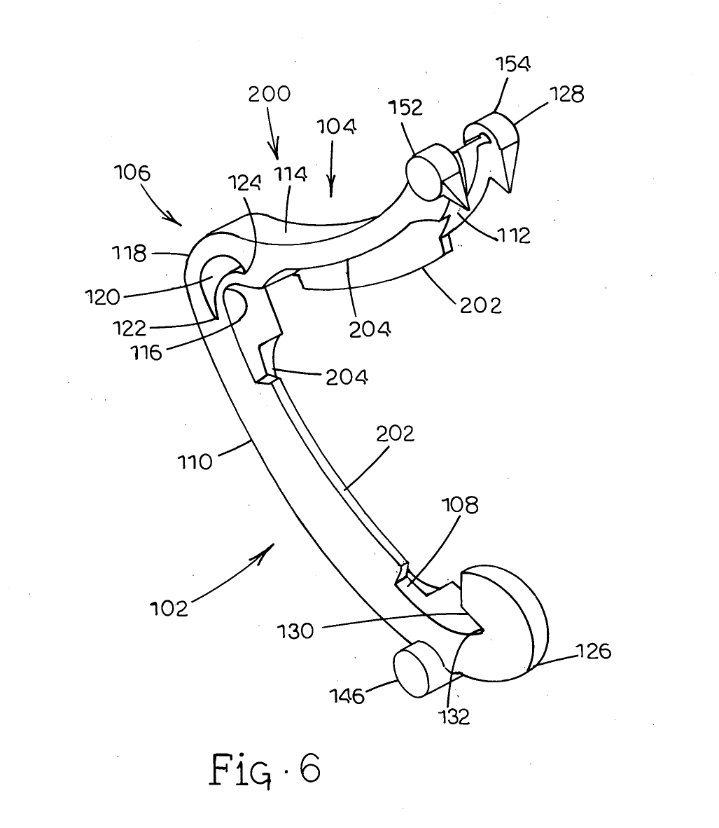

FIGS. 6-10 depict an alternate embodiment of an asymmetric surgical clip 200 in accordance with the invention. Clip 200 bears many similarities to clip 100 described above with reference to FIGS. 1-5. For example, the materials and procedures used to make and apply clip 100 may be used to make and apply clip 200 as well. Similarly, the various features of clip 100 described above are referenced where appropriate in FIGS. 6-10 with respect to clip 200 using the same reference numerals used in FIGS. 1-5.

FIG. 6 is an enlarged perspective view of clip 200. The body of clip 200 includes a first or outer leg, generally designated 102, and a second or inner leg, generally designated 104. First and second legs 102 and 104 are joined at their proximal ends by an integral hinge section, generally designated 106. First and second legs 102 and 104 have complementary arcuate profiles. Thus, first leg 102 has a concave inner surface 108 and a convex outer surface 110, and second leg 104 has a convex inner surface 112 and a concave outer surface 114. Convex inner surface 112 of second leg 104 and concave inner surface 108 of first leg 102 have substantially matching radii of curvature.

Hinge section 106 has a continuous concave inner surface 116 and a continuous convex outer surface 118. Concave inner surface 116 of hinge section 106 joins concave inner surface 108 of first leg 102 and convex inner surface 112 of second leg 104. Convex outer surface 118 of hinge section 106 joins convex outer surface 110 of first leg 102 and concave outer surface 114 of second leg 104. Curved slot 120 is located between curved hinge surfaces 116 and 118, and is positioned closer to inner surface 116 than to outer surface 118. Slot 120 extends completely through hinge section 106 from side to side and its opposite ends 122, 124 extend into the proximal ends of first and second legs 102 and 104, respectively.

First leg 102 transitions to a curved, C-shaped hook section 126 at its distal end. Second leg 104 transitions to a pointed tip section 128 at its distal end. The distal portion of hook section 126 curves inwardly and points generally toward inner surface 116 of hinge 106. The hook section has a transverse beveled surface 130 and a concave inner surface 108 that defines a latching recess 132. The latching recess 132 is adapted to conformally engage tip section 128 in the course of compressing clip 100 into a latched or locked position around a vessel or other tissue.

In accordance with the alternate embodiment of the invention, complementary parts of an interlocking mechanism are formed along the inner surfaces of first leg 102 and second leg 104. In this embodiment, the complementary parts are arranged in a lock-step configuration. A raised lip or tongue 202 protrudes from and is oriented longitudinally along a portion of inner surface 112 of second leg 104. Recessed pocket or groove 204 is formed adjacent to tongue 202 and runs parallel to tongue 202 along inner surface 112 of second leg 104. Another tongue 202 and groove 204 are similarly arranged along inner surface 108 of first leg 102, with tongue 202 of one leg member aligned opposite to groove 204 of the other leg member so as to interlock when clip 100 is closed.

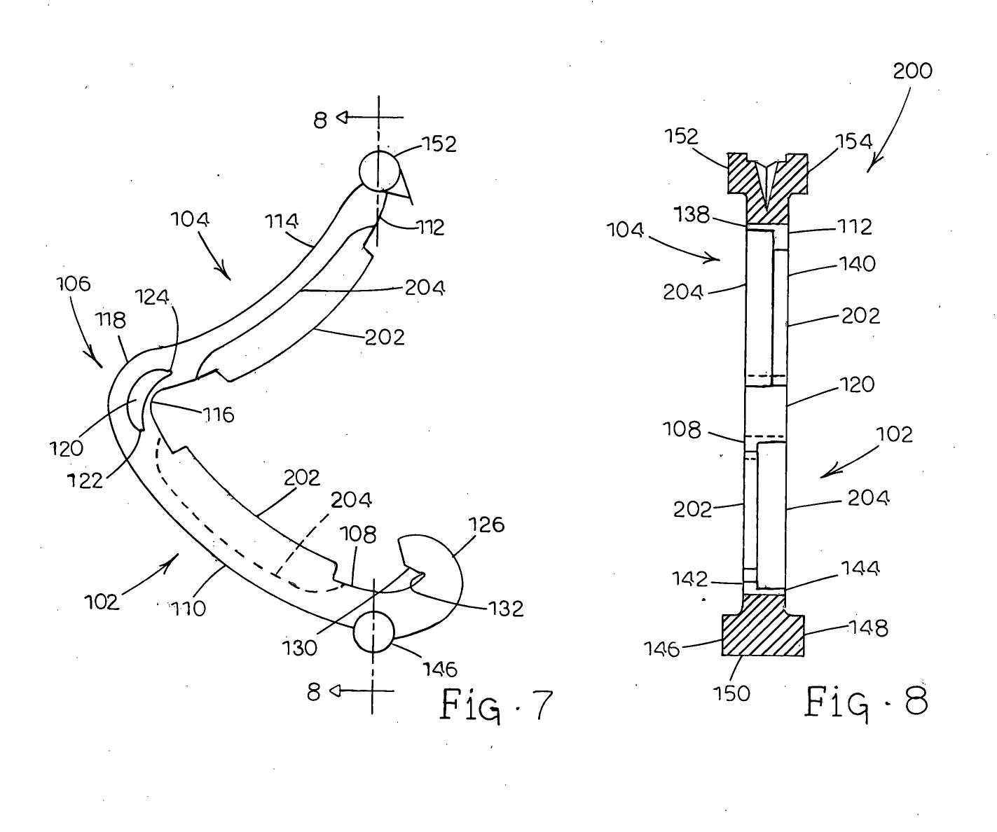

As shown in FIG. 7, which is an enlarged side elevation view of the surgical ligating clip of the present invention, the proximal and distal ends of tongue 202 may be curved, giving tongue 202 a generally oval or elliptical profile. Groove 204 has a profile complementary to tongue 202 and is positioned opposite to tongue 202. Each tongue 202 and groove 204 pair form complementary parts of an interlocking mechanism. Accordingly, when clip 200 is compressed into a latched or locked position, each tongue 202 fits within the opposing groove 204. The curved ends of tongue 202 reduce the likelihood that tissue captured in clip 200 would be damaged by tongue 202. One would appreciate that groove 204 should be larger than tongue 202 to accommodate tongue 202 and the portion of any vessel or tissue captured by clip 200 along tongue 202.

As best shown in FIG. 8, which is a view directed into the open concave side of clip 200 viewed along line 8-8 in FIG. 7, clip 200 has parallel, opposed side surfaces. Tongue 202 may abut one of the side surfaces of a leg and groove 204 may abut the other side surface of the leg. The portion of the side surface co-incident with groove 204 may follow the profile of groove 204, thereby providing an open side for groove 204. The width and length of tongue 202 are smaller than the width and length of groove 204. As noted above, the larger dimensions of groove 202 permit an amount of tissue to be pushed into groove 204 by tongue 202.

Adjacent to the distal end of the first leg 102 and immediately inward of hook section 126, cylindrical bosses 146 and 148 protrude perpendicular to each of the opposed side surfaces 138 and 140. In the illustrated example of clip 100, a bridge section 150 couples bosses 146 and 148 together. As evident in FIG. 8, bosses 146 and 148 project outwardly beyond convex outer surface 110 of first leg 102. At the distal end of second or inner leg 104, cylindrical bosses 152 and 154 protrude perpendicular to each of the opposed side surfaces of inner leg 104 at tip section 128. Bosses 152 and 154 of second leg 104 extend longitudinally forwardly beyond tip section 128.

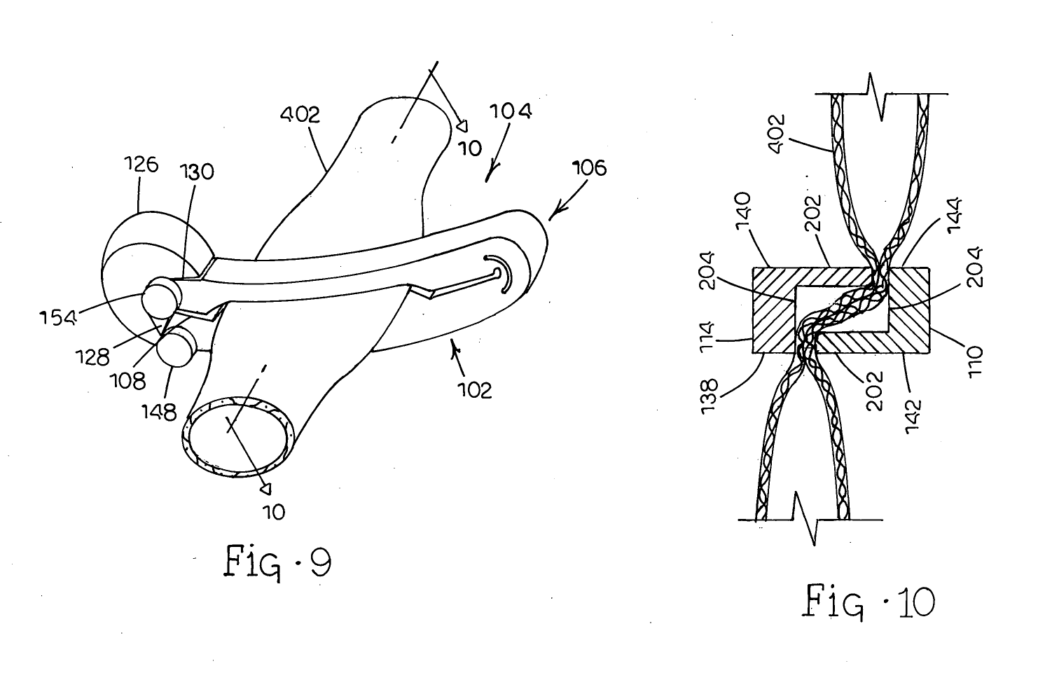

FIG. 9 is an enlarged, perspective view of clip 200 compressed around a portion of a vessel. It should be appreciated that clip 200 may be compressed and latched as described above with reference to clip 100. In the latched condition, tip section 128 is engaged between concave inner surface 108 and beveled surface 130, thereby securely clamping a designated vessel or other tissue between concave inner surface 108 and convex inner surface 112.

FIG. 10 is a cross sectional view of clip 200 engaged around a portion of a vessel viewed along line 10-10 in FIG. 9. In the area where clip 200 is applied to the vessel, each tongue 202 makes contact with a portion of the vessel and pushes the vessel into opposite groove 204. The portion of the vessel between tongues 202 conforms around tongues 202 as the vessel is pushed into grooves 204. Longitudinal movement of the vessel relative to clip 200 is resisted primarily by the portion of the vessel trapped between the medial surfaces of tongues 202.

Prior art clips similar to clip 100 are described in detail in the commonly assigned U.S. Pat. No. 4,834,096 to Oh et al. and U.S. Pat. No. 5,062,846 to Oh et al., the disclosures of which are incorporated herein in their entireties. In addition, a particularly suitable clip is the HEM-O-LOK® clip commercially available from the assignee of the present invention. These clips are currently available in sizes designated “M”, “ML”, “L”, and “XL”. The clip cartridge of the invention described hereinbelow can be adapted to accommodate any sizes of HEM-O-LOK® clips commercially available.

It will be understood that various details of the invention may be changed without departing from the scope of the invention. Furthermore, the foregoing description is for the purpose of illustration only, and not for the purpose of limitation—the invention being defined by the claims.

1. In a polymeric surgical clip comprising first and second leg members joined at their proximal ends by a resilient hinge, each leg member having a vessel clamping inner surface and an opposite outer surface, the vessel clamping inner surface being in opposition to the vessel clamping inner surface of the other leg member, the first leg member terminating at its distal end in a deflectable hook member curved toward the second leg member, the second leg member terminating at its distal end in a locking portion complementary to the hook member whereby when the first and second leg members are moved from an open position to a closed position about the hinge, the hook member deflects about the distal end of the second leg member to lock the clip in a closed position, the hook member having a continuously curved outer surface extending distally from the outer surface of the first leg member, side surfaces and an inner surface; the improvement comprising:
complementary parts of an interlock mechanism formed along a portion of the vessel clamping inner surface of each of the first and second leg members, the complementary parts cooperating when the clip is in the closed position to capture a vessel and impede longitudinal movement of the clip in relation to the vessel.

2. The surgical clip according to claim 1, wherein the interlock mechanism includes a ridge portion formed along a portion of the vessel clamping inner surface of one of the first and second leg members and a groove portion formed along a portion of the vessel clamping inner surface of the other of the first and second leg members.

3. The surgical clip according to claim 2, wherein when the clip is in the closed position, the ridge and groove are aligned.

4. (canceled)

5. The surgical clip according to claim 1, wherein the inner surface of the first leg member has a concave radius of curvature between the hinge and the hook member and the outer surface of the first leg member has a convex radius of curvature, the inner surface of the second leg member has a convex radius of curvature between the hinge and its distal end and the outer surface of the second leg member has a concave radius of curvature between the hinge and its distal end.

6. The surgical clip according to claim 1, wherein the clip comprises bosses coupled to the first and second leg members for engagement with a suitable clip applier for applying the clips, the bosses joined in pairs to opposite sides of the first leg member between the hinge and the hook portion, and to opposite sides of the second leg member at the distal end of the second leg member, the second leg member having sharp pointed members extending from the bosses.

7. The surgical clip according to claim 6, wherein a portion of the pair of bosses joined to the first leg member extend beyond the outer surface of the first leg member to form substantially parallel and spaced apart surfaces which prevent lateral movement of the first and second leg members relative to one another when the clip is in the closed position.

8. In a polymeric surgical clip comprising first and second leg members joined at their proximal ends by a resilient hinge, each leg member having a vessel clamping inner surface and an opposite outer surface, the vessel clamping inner surface being in opposition to the vessel clamping inner surface of the other leg member, the first leg member terminating at its distal end in a deflectable hook member curved toward the second leg member, the second leg member terminating at its distal end in a locking portion complementary to the hook member whereby when the first and second leg members are moved from an open position to a closed position about the hinge, the hook member deflects about the distal end of the second leg member to lock the clip in a closed position, the hook member having a continuously curved outer surface extending distally from the outer surface of the first leg member, side surfaces and an inner surface; the improvement comprising:
a ridge portion formed along a portion of the vessel clamping inner surface of one of the first and second leg members and a groove portion formed along a portion of the vessel clamping inner surface of the other of the first and second leg members, the ridge and groove portions being aligned in opposition to each other and cooperating when the clip is in the closed position to capture a vessel and impede longitudinal movement of the clip in relation to the vessel.

9. The surgical clip according to claim 8, wherein the inner surface of the first leg member has a concave radius of curvature between the hinge and the hook member and the outer surface of the first leg member has a convex radius of curvature, the inner surface of the second leg member has a convex radius of curvature between the hinge and its distal end and the outer surface of the second leg member has a concave radius of curvature between the hinge and its distal end.

10. The surgical clip according to claim 8, wherein the clip comprises bosses coupled to the first and second leg members for engagement with a suitable clip applier for applying the clips, the bosses joined in pairs to opposite sides of the first leg member between the hinge and the hook portion, and to opposite sides of the second leg member at the distal end of the second leg member, the second leg member having sharp pointed members extending from the bosses.

11. A surgical clip comprising:
(a) a first leg and a second leg, each of the legs having an inner vessel-clamping surface and an outer surface, the inner surfaces being positioned in opposition to each other;
(b) a flexible hinge section integrally disposed between and joining the proximal ends of the first and second legs;
(c) a clip-locking mechanism formed by a deflectable hook member formed at the distal end of the first leg member and a complementary locking portion formed at the distal end of the second leg member whereby when the first and second leg members are moved from an open position to a closed position about the hinge, the hook member deflects about the distal end of the second leg member to lock the clip in a closed position;
(d) a protruding ridge formed along a portion of the inner surface of at least one of the first and second legs; and
(e) a groove formed along a portion of at least one of the first and second legs, the groove on one leg being in opposition to the protruding ridge on the other leg such that the ridge fits within the groove when the clip is in the closed position.

12. The surgical clip of claim 11, wherein the protruding ridge is formed along the inner surface of one leg and the groove is formed along the inner surface of the other leg.

13. (canceled)

14. The surgical clip of claim 11, wherein each ridge and groove extends along the inner surface of at least one of the first and second legs from a point near the proximal end of the leg to a point near the distal end of the leg.

15. A surgical clip comprising:
(a) a first leg and a second leg, each of the legs having an inner vessel-clamping surface and an outer surface, the inner surfaces being positioned in opposition to each other;
(b) a flexible hinge section integrally disposed between and joining the proximal ends of the first and second legs;
(c) a clip-locking mechanism formed by a deflectable hook member formed at the distal end of the first leg member and a complementary locking portion formed at the distal end of the second leg member whereby when the first and second leg members are moved from an open position to a closed position about the hinge, the hook member deflects about the distal end of the second leg member to lock the clip in a closed position;
(d) a protruding ridge formed along a portion of the inner surface of one of the first and second legs; and
(e) a groove formed along a portion of the other one of the first and second legs, the groove on one leg being in opposition to the protruding ridge on the other leg such that the ridge fits within the groove when the clip is in the closed position.

16. The surgical clip of claim 15, wherein the ridge and groove extend along the inner surface of their respective one of the first and second legs from a point near the proximal end of the leg to a point near the distal end of the leg.

17. The surgical clip according to claim 15, wherein the inner surface of the first leg member has a concave radius of curvature between the hinge and the hook member and the outer surface of the first leg member has a convex radius of curvature, the inner surface of the second leg member has a convex radius of curvature between the hinge and its distal end and the outer surface of the second leg member has a concave radius of curvature between the hinge and its distal end.

18. The surgical clip according to claim 15, wherein the clip comprises bosses coupled to the first and second leg members for engagement with a suitable clip applier for applying the clips, the bosses joined in pairs to opposite sides of the first leg member between the hinge and the hook portion, and to opposite sides of the second leg member at the distal end of the second leg member, the second leg member having sharp pointed members extending from the bosses.
